(19) United States
(12) Patent Application Publication
(10) Pub. No.: US 2022/0106554 A1
(43) Pub. Date: Apr. 7, 2022

(54) METHOD OF USING TRACK ETCHED MEMBRANES FOR THE FILTRATION OF BIOLOGICAL FLUIDS

(71) Applicant: Repligen Corporation, Waltham, MA (US)

(72) Inventors: Craig Robinson, Hingham, MA (US); Prity Bengani-Lutz, Woburn, MA (US)

(73) Assignee: Repligen Corporation, Waltham, MA (US)

(21) Appl. No.: 17/426,238

(22) PCT Filed: Jan. 30, 2020
(86) PCT No.: PCT/US2020/015814

§ 371 Date: Jul. 28, 2021

Related U.S. Application Data
(60) Provisional application No. 62/798,775, filed on Jan. 30, 2019.

(51) Int. Cl. C12M 1/00; B01D 69/02; B01D 63/08; B01D 39/16; B01D 71/50; B01D 71/64; B01D 71/48; B01D 71/34; B32B 7/12; B32B 27/08; F16L 13/11

(52) U.S. Cl. CPC C12M 47/10; B01D 2315/10; B01D 63/082; B01D 39/1692; B01D 71/50; B01D 71/64; B01D 71/48; B01D 71/34; B32B 7/12; B32B 27/08; F16L 13/11; B01D 2325/04; B01D 2313/04; B01D 2313/146; B01D 2313/143; B01D 2325/021; B01D 69/02

(57) ABSTRACT

Track-etched membranes for filtration are provided. Filtration methods utilizing such membranes and cell culture methods are also provided.

### FIELD OF THE DISCLOSURE

The present disclosure relates to the use of track etched membranes in tangential flow filtration systems.

### BACKGROUND

Filtration is used to separate, clarify, modify, and/or concentrate a fluid solution, mixture, or suspension. It is often a necessary step in the production, processing, and analysis stages of drugs, diagnostics, and chemicals by the biotechnical, pharmaceutical, and medical industries. Filtration may be used to remove a desired compound from a solution, for example, or to remove byproducts, leaving behind a more concentrated medium. These processes can be modified as appropriate by the selection of a variety of filter materials, pore sizes, and/or other filter variables.

Tangential flow filtration (TFF), also known as cross-flow filtration (CFF), is used throughout industry to separate or purify materials fluid suspensions or solutions based on their size or charge differences. In a TFF system, a fluid feed comprising various molecular or particulate species flows into a filtration vessel in a direction parallel to the surface of a semi-permeable membrane. In the filtration vessel, the feed is separated into two component flows: a permeate flow (also referred to as a filtrate) that passes through the membrane and includes certain species from the feed; and a retentate flow which does not pass through the membrane and includes any species that did not pass into the permeate.

In contrast with TFF, conventional direct-flow filtration systems (or dead-end filtration systems) in which fluid flows perpendicular to the surface of the membrane, the accumulation of retained species on the membrane surface occurs much more rapidly which may lead to membrane fouling. TFF systems have several advantages over dead-end systems such as slower buildup of material on the filter surface and lower rates of fouling due to the feed being swept away by tangential flow. This makes them especially suitable for feed streams with high solids content and high viscosity for various applications, including bioprocessing and pharmaceutical applications.

TFF systems are commonly implemented using plate-and-frame or cassette designs. These designs typically incorporate a plurality of flat sheet membranes arranged between external flat plates and manifolds. In use, a fluid feed is passed through the inlet of the manifold into the cassette, and tangentially to the first (upper) surfaces of the membranes. The permeate flow passes through the membranes then through the cassette into a dedicated permeate channel of the manifold, while the retentate does not cross the membrane and passes into a separate retentate channel of the manifold.

Conventionally, cassettes are made by interleaving multiple layers of membranes with pressure-sensitive adhesives (PSA) and screen mesh and, optionally, securing some or all of the layers together, e.g., by encapsulation using a silicone or urethane polymer. TFF cassettes generally include apertures or other features for interfacing with the manifold. Cassette designs can be susceptible to leaking when layers are mis-aligned with one-another or with the manifold interface. Thus, in use, cassettes are often sandwiched between flat plates or gaskets to seal the cassettes against such leakage.

When producing a cell culture, it is often necessary to filter waste from the developing culture. Advancements in biological manufacturing processes now allow for the large scale production of cell cultures, enabling the production of recombinant proteins, virus-like particles (VLP), gene therapy particles, and vaccines, often using process vessel devices. Cell retention devices which remove metabolic waste products and refresh the culture with additional nutrients are widely available. Commonly, this retention is performed using the perfusion of a process vessel culture with membranes using tangential flow filtration or alternating tangential flow (ATF) filtration.

Membranes are often used in cell culture clarification, but their use in applications may be complicated by the potential for fouling of the membranes with cell debris. Fouling, in turn, may cause membranes to retain, rather than pass, the intended product. These are large sized pores typically in the ultrafiltration (UF) and microfiltration (MF) range between 750 kD μm and 0.65 μm. However, there is an ongoing need for improved membranes which resist fouling while allowing filtration of solutions with a high solid content. Further, another issue with traditional membranes is pore clogging. Because of the method of the formation of the pores, the pores are often not linear or particularly even in their size or distribution. This leads to clogging as the filtrate attempts to move through the pore, which hinders further filtration of the solution. Notably, the creation of pores is unpredictable and often results in pores with winding pathways, variable lengths and sizes, and a variety of shapes.

Membranes are also used for monoclonal antibody concentration, protein purification and other ultrafiltration/diafiltration applications. Polarization and membrane fouling both may pose problems for these applications. Due to their uniform pore size and narrow pore size distribution, TE membranes can also find great use in these applications.

One potential material suitable for filtration would be track-etched (TE) membranes. Typically, TE membranes are too thin to be suitable for TFF assembly or processing. This thinness has resulted in membranes which are unable to withstand the operating pressures which are necessary for these types of separations. Regardless, the smooth surface, uniform pore size and path length, and consistent percent porosity of TE membranes provide the incentive to find a solution for their use. Further incentive comes from the level of control possible over the pore size and shape.

Another potential limitation of TE membranes in the past has been low porosity and hence low permeability. However, the membrane surface can be modified using additives to make them more hydrophilic. These additives can include polyvinyl pyrrolidone, amine groups, or other hydrophilic groups.

Chemical stability of membrane cassettes can be an important factor in cassette stability. Sodium hydroxide is often used as a preserving/storage solution (typically for single use applications) as well as cleaning solution (typically for reusable cassettes). With thicker membranes, and improved chemical stability, TE membranes can be used for both single use as well as reusable applications.

### SUMMARY

The present disclosure provides single-use as well as reusable TFF cassettes and methods for making and using them which offer reduced risk of failure and reduced user assembly. One aspect of this disclosure relates to a tangential flow filtration cassette comprising a flexible isolation plate, at least one of a gasket and a filter plate, and a plurality of interleaved layers disposed between the flexible isolation plate and the gasket or filter plate, which layers define at least one membrane, at least one feed channel and at least one filtrate channel. The plurality of interleaved layers may include a filtrate channel spacer which defines an open interior volume bounded by an interior perimeter and which includes one or more fluid ports, a non-flexible feed channel spacer also defining an open interior volume bounded by an interior perimeter and also including one or more fluid ports, and a membrane disposed between the feed and filtrate channel spacers. The layers also optionally include a pressure sensitive adhesive binding together the membrane and one of the filtrate channel spacer and the feed channel spacer, said thin film of pressure sensitive adhesive having a thickness of less than 50% of height of an adjacent channel. The flexible isolation plate may comprise a flexible polymer or a thermoplastic elastomer and is bonded to a first surface of the interleaved stack. The membrane may comprise a plurality of track-etched (TE) membranes, wherein the membranes may comprise a plurality of pores, and the pores may be uniform in size and shape. In various embodiments, the plurality of pores may be uniform in diameter, depth, and/or path length. The average pore size may be about 100 kD to about 30 micron. The TE membrane may have a thickness up to 60 microns. The TE membranes may comprise a plurality of polycarbonate, polyimide, polyvinylidene fluoride or polyester flat sheets. The plurality of pores may have one or more of the following structures: cylinder, cone, cigar, funnel, and hour glass. The TE membranes may have a smooth surface. The cassette may be used in alternating tangential flow filtration. A gasket may be bonded to a second surface of the interleaved stack, which gasket comprising separate fluid ports for the feed and filtrate channels, and a filter plate comprising a fluid manifold is optionally bonded to the gasket such that a feed port of the filter plate is aligned with a feed port of the gasket, and a filtrate port of the filter plate is aligned with a filtrate port of the gasket. In some embodiments, the TFF cassette may include a tab or an engraving on a sidewall of the cassette to identify the TFF cassette by one or more of a stock keeping unit (SKU) number, a lot number, a serial number, a capacity, a number of feed and/or filtrate channels, and a number of membranes. The tab or engraving may include a barcode. In some instances, the TFF cassette may be disposable. The TFF cassette may be reusable. It may comprise a sealed edge. In some cases, the feed channel and/or the filtrate channel may include a screen disposed within the space defined by the channel spacer. The screen may include a woven, non-woven or polymer mesh. The at least one filtrate channel may comprise a filtrate screen disposed within a space defined by the filtrate channel spacer. The filtrate screen may comprise a woven, non-woven, or extruded polymer mesh.

In some aspects, this disclosure may describe a method for the filtration of a cell culture comprising using a cassette. The cassette may comprise a flexible isolation plate, at least one of a gasket and a filter plate, and, disposed between the flexible isolation plate and the gasket or filter plate, a plurality of interleaved layers defining at least one feed channel and at least one filtrate channel, wherein the plurality of interleaved layers may comprise a filtrate channel spacer defining an open interior volume bounded by an inner perimeter and including one or more fluid ports, a non-flexible feed channel spacer defining an open interior volume bounded by an inner perimeter and including one or more fluid ports, a membrane disposed between the filtrate channel spacer and the feed channel spacers, and, optionally, a pressure sensitive adhesive binding together the membrane and one of the filtrate channel spacer and the feed channel spacer, said thin film of pressure sensitive adhesive having a thickness of less than 50% of height of an adjacent channel wherein (a) the flexible isolation plate comprises a flexible polymer or a thermoplastic elastomer and is bonded to a first surface of the interleaved stack and (b) the membrane comprises a plurality of track-etched membranes, wherein the membranes comprise a plurality of pores, and wherein the pores are uniform in size and shape, connecting the cassette housing to a process vessel, connecting the cassette housing to a separation system, circulating a cell culture through the track etched membrane filter and separation system, filtering the cell culture through the membrane filter, collecting the resulting filtrate, and returning the cell culture to the process vessel. In some embodiments, the cell culture may be a mammalian cell culture. The circulation of the cell culture may be performed by alternating tangential flow. The circulation of the cell culture may be performed by tangential flow filtration.

TE membranes have been found to be appropriate for the filtration in these cassettes due to their smooth surface, uniform pore size, consistent porosity, and ability to have a variety of pore dimensions. The production of thicker TE membranes of up to 60 microns allows for their use in filters as they are robust enough to withstand the pressure of the system. A TE membrane (such as that made by it4ip) may be used within a cassette in conjunction with a process vessel and a traditional separation system. When the system is used with alternating tangential flow or tangential flow filtration, the TE membrane results in more effective filtration of the filtrate, leading to greater concentration of the retentate. Further, because the TE membranes can be created with pores much larger than traditional membrane materials (up to 30 microns in size), these filtration methods may be useful for areas outside of bioprocessing. The ability to create consistent, cylindrical (or similarly shaped) pores allows greater movement of the identified filtrate through the pores, preventing clogging of the pores. Unlike the pores of traditional membranes which are unevenly distributed and variable in size, path length, and shape, TE membranes allow for the creation of controlled, deliberate pores with a definitive size, shape, and path length.

### BRIEF DESCRIPTION OF THE DRAWINGS

FIG. 1 depicts scanning electron microscope (SEM) images of a membrane made by phase inversion and another membrane made by track etch process.

FIG. 2 depicts exemplary data comparing the capacity of TE membranes topolyethersulfone membranes made by phase inversion.

FIG. 3 shows the water flux of a TE cassette versus transmembrane pressure values for a track etched membrane according to an embodiment of the present disclosure.

FIG. 4 shows the water flux of a TE cassette before and after flushing with sodium hydroxide.

### DETAILED DESCRIPTION

Overview

TE membranes have been found here to be appropriate for the filtration of cell cultures and other biological separations. Without wishing to be bound by any theory, it is believed that the performance of these membranes is due to their resistance to fouling, as well as the ability to control the pore shape and size, resulting in efficient and effective separations. These characteristics may be advantageous for a number of bioprocessing applications.

The present disclosure focuses on tangential flow filtration. In exemplary systems designed for these applications, a tangential-flow TE membrane is disposed within a filter cassette to define feed/retentate and permeate (also referred to as filtrate) fluid channels separated from one another by the filter element. In this system, the feed/retentate channel is in fluid communication with a bioreactor or other process vessel, by means of a fluid coupling between the process vessel and a feed channel (corresponding to a fluid feed) of the filter housing and, optionally, a return coupling between an outlet of the filter housing (corresponding to a retentate) and the process vessel. Filtration culture systems according to this disclosure which utilize TE membranes in their filter may offer more effective filtration of the filtrate with reduced fouling, leading to greater concentration of the retentate.

The TE membranes of the present disclosure may be made from polycarbonate, polyimide, polyvinylidene fluoride and/or polyester. Without wishing to be bound by any theory, these materials create a smooth surface for the membrane, which may prevent fouling on the surface. These membranes may have a thickness of up to 60 microns and may have pore sizes in the range of 100 kD to 30 micron. Without wishing to be bound by any theory, the thickness of the membranes may allow the membrane to withstand the high operating pressures of the system. The pores within the membrane may be any of a variety of shapes, including cylindrical, cone, cigar, funnel and hour-glass shaped. Without wishing to be bound by any theory, the ability to control the shape of the pores may allow for greater and more specific separation of the solution. The pores may also be uniform in opening diameter, path length, shape, and density. Without wishing to be bound by any theory, the uniformity of opening diameter, path length, shape, and density of the pores may allow for more specific separations and less fouling than traditional membranes. The track-etched membrane sheet contains a plurality of pores that are consistent in size and shape, where the pores are uniform in size and shape. The pores may be larger than 1 micron. Without wishing to be bound by any theory, larger pore sizes may allow for the practice of macro level filtration.

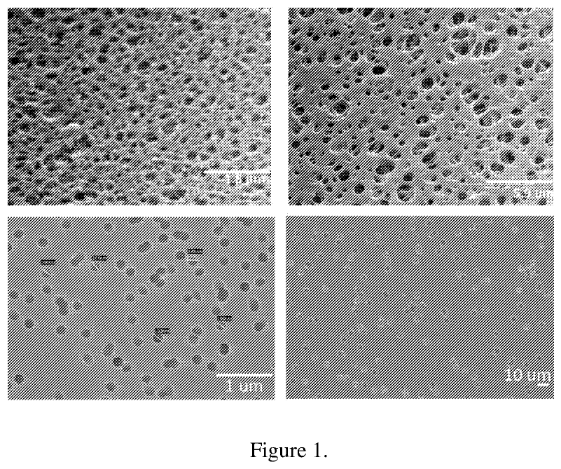

FIG. 1 exemplifies the difference in pore size and shape between PES membranes and TE membranes. The top row shows scanning electron microscope (SEM) images of the surface of 2 different PES membranes made by traditional phase inversion method (magnification 20000× and 5000× respectively). Bottom row shows SEM image of the surface of two different TE membranes (magnification 25000× and 500× respectively).

The filtrate channel spacer defines an open interior volume bounded by an inner perimeter and includes one or more fluid ports. The non-flexible feed channel spacer also defines an open interior volume bounded by an inner perimeter and also includes one or more fluid ports.

A filtration cassette of this disclosure may be used in a variety of small and large-scale applications requiring cross-flow filtration and may be particularly suitable in small and large scale pharmaceutical and biopharmaceutical filtration processes including, but not limited to, the production of vaccines, monoclonal antibodies, and patient-specific treatments.

The cassette of this disclosure generally comprises a flexible isolation plate and either one or both of a gasket and a filter plate. Between the flexible isolation plate and the gasket and/or filter plate are the plurality of interleaved layers creating at least one feed channel and at least one filtrate channel. The plurality of interleaved layers include a filtrate channel spacer, a non-flexible feed channel spacer, a membrane between the filtrate channel spacer and the feed channel spacer, and potentially a pressure sensitive adhesive binding together the membrane and either the filtrate channel spacer and/or the feed channel spacer. The pressure sensitive adhesive has a thickness of less than 50% of the height of the adjacent channel. The flexible isolation plate is made of a flexible polymer or a thermoplastic elastomer and is bonded to the first surface of the interleaved stack.

In the cassette, the gasket may be bonded to a second surface of the interleaved stack and may make up separate fluid ports for the feed and filtrate channels. When a filter plate is bonded to a gasket, the filter plate is a fluid manifold where a feed port of the filter plate is aligned with a feed port of the gasket, and a filtrate port of the filter plate is aligned with a filtrate port of the gasket.

The cassette may have a tab or engraving on a sidewall of the cassette to identify the cassette by a stock keeping unit (SKU) number, a lot number, a serial number, a capacity, a number of feed and/or filtrate channels, and a number of membranes. This tab or engraving may be a barcode.

The cassette may be disposable after use and may also have a sealed edge.

The feed channel of the cassette may have a feed screen within a space defined by the feed channel spacer. This feed screen may be a woven or extruded polymer mesh. The filtrate channel may be a filtrate screen within a space defined by the filtrate channel spacer. The filtrate screen may be a woven or extruded polymer mesh.

Filtration systems according to the present disclosure may comprise track etched membranes, cassette housings, conduits, and other elements that are durable and can be sterilized (e.g., through autoclaving, steam cleaning, gamma irradiation, chemical sterilization, etc.), elements that can be cleaned with commonly used chemical reagents (such as sodium hydroxide) and reused multiple times; alternatively, one or more elements may be single-use and may be disposed of following use.

Also described by this disclosure is a method of use for a system to filter solutions using a TE membrane filter. In one set of embodiments, a TE membrane filter may be used with a cassette housing. The housing may be connected to a process vessel through a first conduit and a filtrate receptacle through a second conduit. A pump may be attached to the system. Activation of the pump may pull liquid through the TE membrane filter cassette, removing the filtrate and retaining the retentate. In some embodiments, the solution is a biological solution and in other embodiments, the solution is a cell culture. The cell culture may be mammalian and the circulation may be performed by alternating tangential flow or tangential flow filtration.

### EXAMPLES

Certain principles of this disclosure are further illustrated by the following examples:

### Example 1

A flat sheet TE membrane was fabricated into 100 cm² cassette with an “E screen” spacer (i.e. coarse feed spacer from Repligen Corporation, Waltham Mass.). The cassette was flushed with DI water, the channels emptied and an air integrity test was performed to test if the cassette is integral and fit for use. At 3 psi, which is in the typical test condition range for MF membranes, the air flow was less than 1.2 ml/min indicating that the cassette was integral.

### Example 2

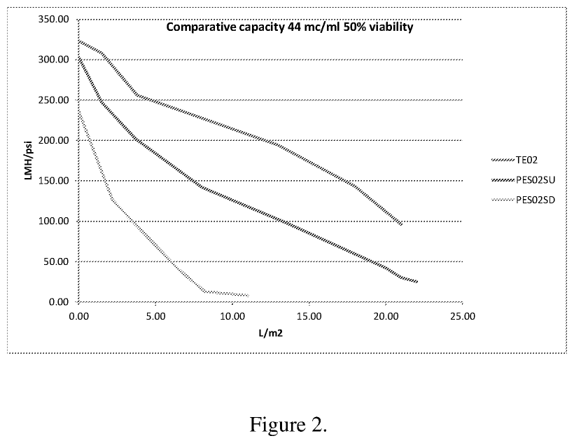

A flat sheet TE membrane with a pore size of 0.2 micron was fabricated into 100 cm² cassettes with “E-screen” (i.e. coarse feed spacer from Repligen) (TE02). Its performance was compared to cassettes made with 0.2 micron polyethersulfone (PES) phase inversion membranes in skin up (PES02SU) and skin down (PES02SD) orientation using two different Chinese Hamster Ovary (CHO) cell culture feed streams. This resulted in the positive result seen in FIG. 2. TE membranes showed higher permeability (LMH/psi), higher capacity (L/m²) and higher sieving than PES membranes of similar pore size. Protein sieving of TE02 membrane was significantly higher and stayed constant at 78%. PES02SU showed a sharp decline in sieving going from 68% at start to 34% by the end, i.e. less than half the sieving obtained using TE02 cassettes.

In comparison to phase inversion membranes (PES02SU and PES02SD), the pressure drop of TE02 was the lowest indicating higher resistance to fouling. The pressure drop at the start were similar for all 3 membranes, at 0.22-0.24 psi. Towards the end, the pressure drop of TE02 membrane was only 0.88 psi, while that of PES02SU was 1.6 psi (i.e. twice as high), indicating a greater degree of fouling.

### Example 3

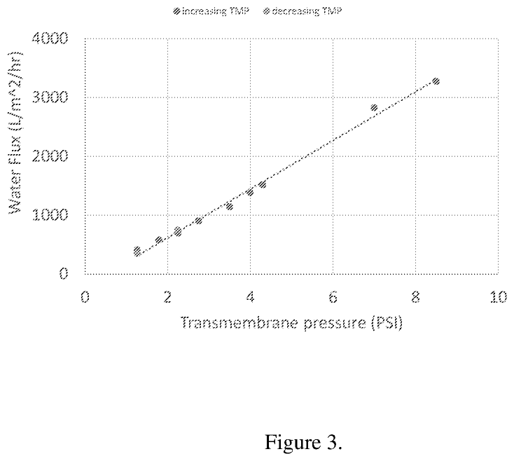

To test the ability of cassettes made of TE membranes to withstand the pressures needed for TFF applications, water was passed through a 100 cm′ TE cassette and the flux was measured at various transmembrane pressures (TMP). Tests were conducted by incrementally increasing the TMP values from low to high i.e. 1 to 8.5 psi, and measuring the water flux at each TMP. To ensure that the membrane was not damaged after testing at high pressures, the test was run by decreasing the TMP from high to low values from 8.5 psi to 2.2 and 1.2 psi. As shown in FIG. 3, there was no significant difference in the water fluxes measured in the either direction, indicating robust pressure stability.

### Example 4

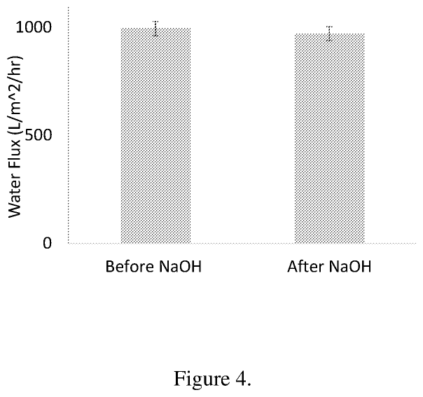

To test the chemical stability of cassettes made with TE membranes, 100 cm′ cassettes were flushed with 0.2 N sodium hydroxide solution using 2 liters per m² of membrane area. Cassettes were flushed for 30 minutes and the water flux before and after was measured at a TMP of 3 psi. The water flux before and after the chemical cleaning was nearly identical, as seen in FIG. 4 indicating good chemical stability to reagents commonly used for cleaning and storing cassettes, and potential for making reusable cassettes.

1. A tangential flow filtration (TFF) cassette, comprising:
a flexible isolation plate;
at least one of a gasket and a filter plate; and
disposed between the flexible isolation plate and the gasket or filter plate, a plurality of interleaved layers defining at least one membrane, at least one feed channel and at least one filtrate channel, the plurality of interleaved layers comprising:
a filtrate channel spacer defining an open interior volume bounded by an inner perimeter and including one or more fluid ports;
a non-flexible feed channel spacer defining an open interior volume bounded by an inner perimeter and including one or more fluid ports;
a membrane disposed between the filtrate channel spacer and the feed channel spacers; and, optionally,
a pressure sensitive adhesive binding together the membrane and one of the filtrate channel spacer and the feed channel spacer, said thin film of pressure sensitive adhesive having a thickness of less than 50% of height of an adjacent channel;
wherein (a) the flexible isolation plate comprises a flexible polymer or a thermoplastic elastomer and is bonded to a first surface of the interleaved stack and (b) the membrane comprises a plurality of track-etched (TE) membranes, wherein the membranes comprise a plurality of pores, and wherein the pores are uniform in size and shape.

2. The cassette of claim 1, wherein the plurality of pores are uniform in diameter, depth, and/or path length.

3. The cassette of claim 1, wherein the average pore size is about 100 kD to about 30 micron.

4. The cassette of claim 1, wherein the TE membrane has a thickness up to 60 microns.

5. The cassette of claim 1, wherein the track-etched membranes comprise a plurality of polycarbonate, polyimide, polyvinylidene fluoride or polyester flat sheets.

6. The cassette of claim 1, wherein the plurality of pores may have one or more of the following structures: cylinder, cone, cigar, funnel and hour glass.

7. The cassette of claim 1, wherein the track-etched membranes have a smooth surface.

8. The cassette of claim 1, wherein the cassette is used in alternating tangential flow filtration.

9. The cassette of claim 1, wherein a gasket is bonded to a second surface of the interleaved stack, the gasket comprising separate fluid ports for the feed and filtrate channels.

10. The cassette of claim 1, wherein a filter plate is bonded to the gasket, the filter plate comprising a fluid manifold wherein a feed port of the filter plate is aligned with a feed port of the gasket, and a filtrate port of the filter plate is aligned with a filtrate port of the gasket.

11. The cassette of claim 1, further comprising a tab or an engraving on a sidewall of the cassette to identify the TFF cassette by one or more of a stock keeping unit (SKU) number, a lot number, a serial number, a capacity, a number of feed and/or filtrate channels, and a number of membranes.

12. The cassette of claim 1, wherein the tab or engraving comprises a barcode.

13. The cassette of claim 1, wherein the TFF cassette is disposable after use.

14. The cassette of claim 1, wherein the TFF cassette is reusable.

15. The cassette of claim 1, wherein the TFF cassette comprises a sealed edge.

16. The cassette of claim 1, wherein the at least one feed channel comprises a feed screen disposed within a space defined by the feed channel spacer.

17. The cassette of claim 16, wherein the feed screen comprises a woven, non-woven or extruded polymer mesh.

18. The cassette of claim 16, wherein the at least one filtrate channel comprises a filtrate screen disposed within a space defined by the filtrate channel spacer.

19. The cassette of claim 18, wherein the filtrate screen comprises a woven, non-woven or extruded polymer mesh.

20. A method for the filtration of a cell culture, comprising:
using a cassette, wherein the cassette comprises:
a flexible isolation plate;
at least one of a gasket and a filter plate; and
disposed between the flexible isolation plate and the gasket or filter plate, a plurality of interleaved layers defining at least one feed channel and at least one filtrate channel, the plurality of interleaved layers comprising:
a filtrate channel spacer defining an open interior volume bounded by an inner perimeter and including one or more fluid ports;
a non-flexible feed channel spacer defining an open interior volume bounded by an inner perimeter and including one or more fluid ports;
a membrane disposed between the filtrate channel spacer and the feed channel spacers; and, optionally,
a pressure sensitive adhesive binding together the membrane and one of the filtrate channel spacer and the feed channel spacer, said thin film of pressure sensitive adhesive having a thickness of less than 50% of height of an adjacent channel;
wherein (a) the flexible isolation plate comprises a flexible polymer or a thermoplastic elastomer and is bonded to a first surface of the interleaved stack and (b) the membrane comprises a plurality of track-etched membranes, wherein the membranes comprise a plurality of pores, and wherein the pores are uniform in size and shape;
connecting the cassette housing to a process vessel;
connecting the cassette housing to a separation system;
circulating a cell culture through the track etched membrane filter and separation system;
filtering the cell culture through the membrane filter;
collecting the resulting filtrate; and
returning the cell culture to the process vessel.

21. The method of claim 20, wherein the cell culture is a mammalian cell culture.

22. The method of claim 20, wherein the circulation of the cell culture is performed by alternating tangential flow.

23. The method of claim 20, wherein the circulation of the cell culture is performed by tangential flow filtration.
